(19) United States
(12) Patent Application Publication
(10) Pub. No.: US 2026/0240258 A1
(43) Pub. Date: Aug. 20, 2026

(54) AEROSOL GENERATION SYSTEM

(71) Applicant: JAPAN TOBACCO INC., Tokyo (JP)

(72) Inventors: Hirofumi MATSUMOTO, Tokyo (JP); Takao ARADACHI, Tokyo (JP); Manabu YAMADA, Tokyo (JP)

(73) Assignee: JAPAN TOBACCO INC., Tokyo (JP)

(21) Appl. No.: 19/473,155

(22) PCT Filed: May 23, 2023
(86) PCT No.: PCT/JP2023/019176

§ 371 Date: Oct. 7, 2025

(51) Int. Cl. A24F 40/57; A24F 40/465; A24F 40/60; A61M 15/06; H05B 6/06; H05B 6/10

(52) U.S. Cl. CPC A24F 40/57; A24F 40/465; A24F 40/60; H05B 6/06; H05B 6/108; A61M 15/06

(57) ABSTRACT

An aerosol-generating system comprising: an accommodating portion for accommodating a substrate containing an aerosol source; a resonant circuit comprising an electromagnetic induction source for inductively heating a susceptor arranged thermally adjacent to the aerosol source of the substrate accommodated in the accommodating portion; and a control unit for controlling operation of the resonant circuit so as to implement processing to generate an aerosol using the substrate, wherein the control unit repeatedly switches an operating mode of the resonant circuit to a first operating mode or a second operating mode in the processing to generate an aerosol using the substrate, the first operating mode comprises causing the resonant circuit to operate at a frequency included in a first frequency band, the second operating mode comprises causing the resonant circuit to operate at a frequency included in a second frequency band different from the first operating band.

### TECHNICAL FIELD

The present disclosure relates to an aerosol-generating system.

### BACKGROUND ART

Inhalation devices that generate substances to be inhaled by a user are in widespread use. For example, an inhalation device employs an aerosol source for generating an aerosol, and a substrate including a flavor source or the like for imparting a flavor component to the generated aerosol, to generate an aerosol to which the flavor component has been imparted. The user can enjoy the flavor by inhaling the aerosol to which the flavor component has been imparted, generated by the inhalation device. The action by which the user inhales the aerosol will also be referred to below as “puffing” or a “puffing action”. Examples of devices classified as inhalation devices that may be cited include those used in place of cigarettes, such as electronic cigarettes and heated tobacco, and also nebulizers, etc. used for medical purposes. It should be noted that an electronic cigarette is an inhalation device of the type which generates an aerosol by atomizing a liquid aerosol source. Heated tobacco is an inhalation device of the type which generates an aerosol by heating a solid containing an aerosol source.

In recent years, inductively heated inhalation devices have been developed which inductively heat a susceptor, heating the aerosol source via the susceptor and thereby generating an aerosol. For example, PTL 1 below discloses technology for estimating the temperature of the susceptor based on frequency characteristics during induction heating.

### CITATION LIST

### Patent Literature

PTL 1: JP 2020-516014 A

### SUMMARY OF INVENTION

### Technical Problem

However, the technology disclosed in PTL 1 has only recently been developed, and there is still room for improvement in various aspects.

The present disclosure was devised in view of the problems above, and the objective of the present disclosure lies in providing an arrangement capable of further improving the quality of experience for a user employing an induction-heating inhalation device.

### Solution to Problem

In order to solve the problem above, one aspect of the present invention provides an aerosol-generating system comprising: an accommodating portion for accommodating a substrate containing an aerosol source; a resonant circuit comprising an electromagnetic induction source for inductively heating a susceptor arranged thermally adjacent to the aerosol source of the substrate accommodated in the accommodating portion; and a control unit for controlling operation of the resonant circuit so as to implement processing to generate an aerosol using the substrate, wherein the control unit repeatedly switches an operating mode of the resonant circuit to a first operating mode or a second operating mode in the processing to generate an aerosol using the substrate, the first operating mode comprises causing the resonant circuit to operate at a frequency included in a first frequency band, the second operating mode comprises causing the resonant circuit to operate at a frequency included in a second frequency band, and the first frequency band and the second frequency band are separate from each other.

The second frequency band may include a resonant frequency of the resonant circuit when a temperature of the susceptor is the highest temperature expected in the processing to generate an aerosol using the substrate.

The second frequency band may include a resonant frequency of the resonant circuit when a temperature of the susceptor is the lowest temperature expected in the processing to generate an aerosol using the substrate.

The first frequency band may be a higher frequency band or a lower frequency band than the second frequency band.

In the first operating mode, the control unit may use one fixed frequency included in the first frequency band.

In the second operating mode, the control unit may use one fixed frequency included in the second frequency band.

The control unit may switch the first frequency band to a higher frequency band or a lower frequency band than the second frequency band, based on electrical characteristics of the resonant circuit acquired in the first operating mode.

The control unit may control operation of the resonant circuit in the second operating mode based on electrical characteristics of the resonant circuit acquired in the first operating mode.

The resonant circuit may further comprise a capacitor, and the control unit may control operation of the resonant circuit in the second operating mode based on a voltage of the capacitor acquired in the first operating mode.

The control unit may estimate a temperature of the susceptor based on the voltage of the capacitor acquired in the first operating mode, and may control operation of the resonant circuit in the second operating mode based on the estimated temperature of the susceptor and a predefined target temperature of the susceptor.

In the second operating mode, the control unit may control a duty ratio of a voltage applied to the resonant circuit.

Power supplied to the resonant circuit in the first operating mode may be smaller than power supplied to the resonant circuit in the second operating mode.

A duration of the first operating mode may be shorter than a duration of the second operating mode.

The control unit may variably set the duration of the second operating mode.

The aerosol-generating system may further comprise the substrate, and the substrate may further contain the susceptor.

### Advantageous Effects of Invention

The present disclosure as described above provides an arrangement capable of further improving the quality of experience for a user employing an induction-heating inhalation device.

### BRIEF DESCRIPTION OF DRAWINGS

FIG. 1 is a schematic diagram schematically showing a configuration example of an inhalation device.

FIG. 2 schematically shows an example of a resonant circuit of an inhalation device according to the embodiment.

FIG. 3 is a diagram to illustrate an example of switching of an operating mode of the inhalation device according to the embodiment.

FIG. 4 is a graph showing a relationship between capacitor voltage and drive frequency in a resonant circuit with a resonant frequency in the 300-400 kHz band.

FIG. 5 is a graph showing a relationship between capacitor voltage and susceptor temperature when a resonant circuit with a resonant frequency in the 300-400 kHz band is operated at the resonant frequency or a frequency outside the resonant frequency.

FIG. 6 is a graph showing a relationship between capacitor voltage and drive frequency in a resonant circuit with a resonant frequency in the 1000-1100 kHz band.

FIG. 7 is a graph showing a relationship between capacitor voltage and susceptor temperature when a resonant circuit with a resonant frequency in the 1000-1100 kHz band is operated at the resonant frequency or a frequency outside the resonant frequency.

FIG. 8 is a flowchart showing an example of a flow of processing implemented by the inhalation device according to the embodiment.

### DESCRIPTION OF EMBODIMENTS

Preferred embodiments of the present disclosure will be described in detail below with reference to the appended drawings. It should be noted that components having substantially the same functional configuration will be assigned the same reference numbers in the description and drawings to avoid giving a duplicate description.

### 1. Configuration Example of Inhalation Device

An inhalation device is a device for generating a substance to be inhaled by a user. Hereinafter, the substance generated by the inhalation device will be described as being an aerosol. Additionally, the substance generated by the inhalation device may be a gas.

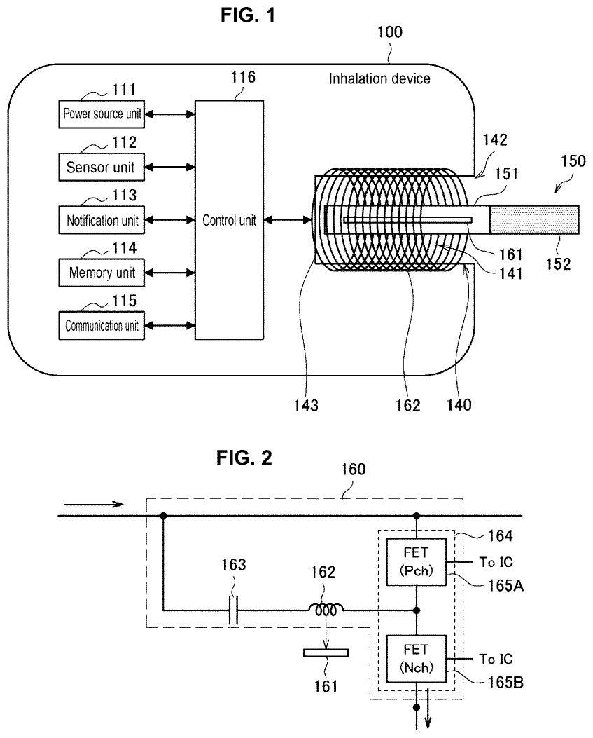

FIG. 1 is a schematic diagram schematically showing a configuration example of an inhalation device. As shown in FIG. 1, an inhalation device 100 according to this configuration example comprises a power source unit 111, a sensor unit 112, a notification unit 113, a memory unit 114, a communication unit 115, a control unit 116, an accommodating portion 140, and an electromagnetic induction source 162.

The power source unit 111 stores electrical power. The power source unit 111 then supplies the electrical power to each component of the inhalation device 100 in accordance with control performed by the control unit 116. The power source unit 111 may be configured by a rechargeable battery such as a lithium ion secondary battery, for example. The power source unit 111 may supply a direct current to other components. Alternatively, the power source unit 111 may supply an alternating current converted by an inverter circuit to the other components.

The sensor unit 112 acquires various types of information relating to the inhalation device 100. As an example, the sensor unit 112 is configured by a pressure sensor such as a condenser microphone, a flow rate sensor or a temperature sensor, etc., and acquires values associated with inhalation by a user. As another example, the sensor unit 112 is configured by an input device, such as a button or switch, for accepting input of information from the user.

The notification unit 113 notifies the user of information. The notification unit 113 is configured by a light-emitting device which emits light, a display device which displays images, a sound output device which outputs sound, or a vibration device which vibrates, etc., for example.

The memory unit 114 stores various types of information for operation of the inhalation device 100. The memory unit 114 is configured by a non-volatile storage medium such as a flash memory, for example.

The communication unit 115 is a communication interface capable of performing communication conforming to any wired or wireless communication standard. Examples of communication standards that may be used include standards that employ Wi-Fi (registered trademark), Bluetooth (registered trademark), BLE (Bluetooth Low Energy) (registered trademark), NFC (Near-Field Communication), or LPWA (Low Power Wide Area), for example.

The control unit 116 functions as an arithmetic processing device and a control device, and controls overall operation within the inhalation device 100 in accordance with various programs. The control unit 116 is realized by a CPU (central processing unit) or an electronic circuit such as a microprocessor, for example.

The accommodating portion 140 has an internal space 141, and holds a stick-type substrate 150 while accommodating a portion of the stick-type substrate 150 in the internal space 141. The accommodating portion 140 has an opening 142 allowing the internal space 141 to communicate with the outside, and accommodates the stick-type substrate 150 which has been inserted into the internal space 141 from the opening 142. For example, the accommodating portion 140 is a cylindrical body comprising the opening 142 and a bottom portion 143 serving as a bottom surface, and defines a columnar internal space 141. An air flow path for supplying air to the internal space 141 is connected to the accommodating portion 140. An air inflow hole, which is an inlet for air into the air flow path, is disposed in a side surface of the inhalation device 100, for example. An air outflow hole, which is an outlet for air from the air flow path to the internal space 141, is disposed in the bottom portion 143, for example.

The stick-type substrate 150 comprises a substrate portion 151 and a mouthpiece portion 152. The substrate portion 151 contains an aerosol source. The aerosol source includes a tobacco-derived or non-tobacco-derived flavor component. If the inhalation device 100 is a medical inhaler such as a nebulizer, the aerosol source may include a drug. The aerosol source may, for example, be a liquid such as water or a polyhydric alcohol, for example glycerol or propylene glycol, containing the tobacco-derived or non-tobacco-derived flavor component, or may be a solid including the tobacco-derived or non-tobacco-derived flavor component. In a state in which the stick-type substrate 150 is held in the accommodating portion 140, at least part of the substrate portion 151 is accommodated in the internal space 141, and at least part of the mouthpiece portion 152 protrudes from the opening 142. Then, when the user holds the mouthpiece portion 152 protruding from the opening 142 in their mouth and inhales, air flows into the internal space 141 via the air flow path, which is not illustrated in the drawings, and reaches the inside of the user's mouth together with the aerosol generated from the substrate portion 151.

The stick-type substrate 150 further comprises a susceptor 161. The susceptor 161 generates heat by electromagnetic induction. The susceptor 161 is made of a conductive material, such as a metal. It is also desirable for the susceptor 161 to be magnetic. As an example, the susceptor 161 may be configured as a metal plate or a metal rod. The susceptor 161 is arranged thermally adjacent to the aerosol source. That is, the susceptor 161 is disposed in a position at which heat generated in the susceptor 161 is transferred to the aerosol source. In the example shown in FIG. 1, the susceptor 161 is included in the substrate portion 151 of the stick-type substrate 150. Moreover, the configuration may be such that the susceptor 161 cannot be touched from the outside of the stick-type substrate 150. For example, the susceptor 161 may run through a central part of the stick-type substrate 150 and may not run close to the outer periphery.

The electromagnetic induction source 162 inductively heats the susceptor 161. The electromagnetic induction source 162 generates a fluctuating magnetic field (more specifically, an alternating magnetic field) when an alternating current is applied thereto. The electromagnetic induction source 162 is disposed in a position at which the generated fluctuating magnetic field overlaps the internal space 141 of the accommodating portion 140, more specifically a position at which the fluctuating magnetic field overlaps the susceptor 161 of the stick-type substrate 150 accommodated in the accommodating portion 140. The electromagnetic induction source 162 comprises, for example, a coiled conductor, and is disposed so as to be wound around the outer periphery of the accommodating portion 140. Thus, when a fluctuating magnetic field is generated in a state in which the stick-type substrate 150 is accommodated in the accommodating portion 140, the fluctuating magnetic field generated from the electromagnetic induction source 162 penetrates the susceptor 161 located in the internal space 141 of the accommodating portion 140 and inductively heats the susceptor 161. More specifically, eddy current losses occur in the susceptor 161, and if the susceptor 161 is magnetic, magnetic hysteresis losses also occur in the susceptor 161, causing the temperature of the susceptor 161 to increase. The aerosol source contained in the stick-type substrate 150 is then heated and atomized by the inductively heated susceptor 161, generating an aerosol. As an example, electricity may be supplied to the electromagnetic induction source 162 when the sensor unit 112 detects that the user has started inhaling and/or that predetermined information has been input. The supply of electricity to the electromagnetic induction source 162 may then be stopped when the sensor unit 112 detects that the user has finished inhaling and/or that predetermined information has been input.

A configuration example of the inhalation device 100 has been described above. The inhalation device 100 is, of course, not limited to the configuration described above, and may adopt various configurations, such as those illustrated below by way of example.

The susceptor 161 may be provided in the inhalation device 100, instead of being contained in the stick-type substrate 150. As an example, the inhalation device 100 may comprise a susceptor 161 arranged outside the internal space 141. Specifically, the accommodating portion 140 may be made of a conductive and magnetic material, and may also function as the susceptor 161. The accommodating portion 140 serving as the susceptor 161 contacts the outer periphery of the substrate portion 151 and may therefore be thermally adjacent to the aerosol source contained in the substrate portion 151. As another example, the inhalation device 100 may comprise a susceptor 161 arranged inside the internal space 141. Specifically, the susceptor 161 configured in a blade-like shape may be arranged so as to protrude into the internal space 141 from the bottom portion 143 of the accommodating portion 140. When the stick-type substrate 150 is inserted into the internal space 141 of the accommodating portion 140, the blade-like susceptor 161 pierces the substrate portion 151 of the stick-type substrate 150 and is inserted inside the stick-type substrate 150. This allows the blade-like susceptor 161 to be thermally adjacent to the aerosol source contained in the substrate portion 151.

### 2. Technical Features

### (1) Configuration of Resonant Circuit

FIG. 2 schematically shows an example of a resonant circuit of the inhalation device 100 according to the embodiment. As shown in FIG. 2, the inhalation device 100 according to the embodiment comprises a resonant circuit 160 which includes the electromagnetic induction source 162 and a capacitor 163.

In the example shown in FIG. 2, the resonant circuit 160 is a series resonant circuit in which the electromagnetic induction source 162 and the capacitor 163 are connected in series. The electromagnetic induction source 162 is what is known as an induction coil. As shown in FIG. 2, the resonant circuit 160 may be an LC resonant circuit which includes the electromagnetic induction source 162 and the capacitor 163. However, in a state in which the stick-type substrate 150 containing the susceptor 161 is accommodated in the accommodating portion 140, the susceptor 161 functions essentially as a resistor in the resonant circuit 160 when the susceptor 161 is inductively heated by the electromagnetic induction source 162. The resonant circuit 160 may therefore also be seen as constituting an RLC resonant circuit which includes the susceptor 161.

As shown in FIG. 2, the resonant circuit 160 includes an inverter circuit 164. The inverter circuit 164 is a half-bridge inverter including two FETs (field-effect transistors) 165 (165A and 165B). For example, the FET 165A is a P-channel FET, and the FET 165B is an N-channel FET. The inverter circuit 164 converts the supplied direct current to alternating current by repeatedly switching ON/OFF states of the two FETs 165. The alternating current converted by the inverter circuit 164 is supplied to the electromagnetic induction source 162 and the capacitor 163.

As shown in FIG. 2, the two FETs 165 are switched ON/OFF based on control performed by an IC (integrated circuit) corresponding to the control unit 116. The control unit 116 then controls a drive frequency of the resonant circuit 160, i.e., the frequency of the alternating current supplied to the electromagnetic induction source 162 and the capacitor 163, by controlling the ON/OFF states of the two FETs 165.

Note that the drive frequency of the resonant circuit 160 may be 100 kHz-7000 kHz, for example. The drive frequency of the resonant circuit 160 may preferably be 300 kHz-2000 kHz, and may even more preferably be 500 kHz-1000 kHz. The resonant frequency of the resonant circuit 160 is preferably within the available range of drive frequencies of the resonant circuit 160.

### (2) Heating Profile

Processing implemented by the control unit 116 to generate an aerosol using one stick-type substrate 150 comprises controlling operation of the resonant circuit 160 based on a heating profile. The heating profile is control information for controlling the temperature at which the aerosol source is heated. The heating profile may be control information for controlling the temperature of the susceptor 161. As an example, the heating profile may include a target value of the temperature of the susceptor 161 (also referred to below as the “target temperature”). The target temperature may vary according to the elapsed time since the start of heating, in which case the heating profile includes information defining a time-series transition of the target temperature.

The control unit 116 controls operation of the resonant circuit 160 so that the real temperature (also referred to below as the “actual temperature”) of the susceptor 161 transitions in a similar way to the time-series transition of the target temperature defined in the heating profile. As a result, an aerosol is generated in accordance with the planned heating profile. The heating profile is typically designed such that, when the user inhales the aerosol generated from the stick-type substrate 150, the flavor tasted by the user is optimized. The flavor tasted by the user can therefore be optimized by controlling operation of the resonant circuit 160 based on the heating profile.

The heating profile may include one or more combinations of elapsed time since heating started and target temperature to be reached at the relevant elapsed time. The control unit 116 then controls the temperature of the susceptor 161 based on a deviation between the current actual temperature and the target temperature in the heating profile corresponding to the current elapsed time since heating started. The temperature control of the susceptor 161 can be realized by known feedback control, for example. In the feedback control, the control unit 116 controls operation of the resonant circuit 160 based on the difference between the actual temperature and the target temperature, etc.

The feedback control may be, for example, PID control (Proportional-Integral-Differential Controller). The control unit 116 may cause power from the power source unit 111 to be supplied to the resonant circuit 160 in the form of pulses by pulse width modulation (PWM) or pulse frequency modulation (PFM). In that case, the control unit 116 can control the temperature of the susceptor 161 by adjusting the duty ratio of the power pulses in the feedback control. Alternatively, the control unit 116 may perform simple ON-OFF control. For example, the control unit 116 may supply power to the resonant circuit 160 until the actual temperature of the susceptor 161 reaches the target temperature, and may interrupt the supply of power to the resonant circuit 160 when the actual temperature has reached the target temperature.

The period from the start to the end of the processing to generate an aerosol using the stick-type substrate 150 is also referred to hereinafter as a heating session. In other words, a heating session is a period of time during which operation of the resonant circuit 160 is controlled on the basis of the heating profile. The beginning of the heating session is the timing at which heating based on the heating profile is started. The end of the heating session is a timing at which a sufficient amount of aerosol is no longer being generated. The heating session comprises a preheating period and a puffing-possible period following the preheating period. The puffing-possible period is the period of time during which a sufficient amount of aerosol is expected to be generated. The preheating period is the period from when heating is started until the puffing-possible period is started. Heating performed in the preheating period is also referred to as preheating.

### (3) Switching of Operating Mode

The control unit 116 repeatedly switches the operating mode of the resonant circuit 160 to a temperature estimation mode or a heating mode in the processing to generate an aerosol using the stick-type substrate 150, that is, in heating processing based on the heating profile. The temperature estimation mode is an operating mode for estimating the temperature of the susceptor 161, and is an example of a first operating mode. The heating mode is an operating mode for heating the susceptor 161, and is an example of a second operating mode. This configuration allows the temperature of the susceptor 161 to be controlled while the temperature of the susceptor 161 is monitored.

The control unit 116 controls operation of the resonant circuit 160 in the heating mode based on electrical characteristics of the resonant circuit 160 acquired in the temperature estimation mode. Electrical characteristics of the resonant circuit 160 as referred to here are electrical characteristics corresponding to the temperature of the susceptor 161. This configuration allows the temperature of the susceptor 161 to be controlled while the temperature of the susceptor 161 is monitored.

The control unit 116 controls operation of the resonant circuit 160 in the heating mode based on a voltage of the capacitor 163 acquired in the temperature estimation mode. More specifically, the control unit 116 estimates the temperature of the susceptor 161 based on the voltage of the capacitor 163 acquired in the temperature estimation mode. The control unit 116 then controls operation of the resonant circuit 160 in the heating mode based on the estimated temperature of the susceptor 161. As will be described later, the voltage (especially the maximum value of AC voltage) of the capacitor 163 according to the embodiment has a strong correlation with the temperature of the susceptor 161. The control unit 116 therefore estimates the temperature of the susceptor 161 by referencing the acquired voltage of the capacitor 163 in a lookup table defining correspondences between voltages of the capacitor 163 and estimated temperature values of the susceptor 161. This lookup table is stored in the memory unit 114. This configuration allows the temperature of the susceptor 161 to be controlled while the temperature of the susceptor 161 is very accurately estimated.

The ability to estimate the temperature of the susceptor 161 based on the voltage of the capacitor 163 means that the temperature of the susceptor 161 can be estimated without providing a separate temperature sensor, etc. in contact with the susceptor 161. This is particularly effective in configurations where it is difficult to measure the temperature of the susceptor 161 by placing a temperature sensor in contact with the susceptor 161, such as when the susceptor 161 is built into the stick-type substrate 150.

The control unit 116 controls operation of the resonant circuit 160 in the heating mode based on the estimated temperature of the susceptor 161 and a predefined target temperature of the susceptor 161. This configuration allows the temperature of the susceptor 161 to transition as defined in the heating profile. The quality of the user experience can be improved as a result.

In the heating mode, the control unit 116 may control a duty ratio of the voltage applied to the resonant circuit 160. More specifically, in the heating mode, the control unit 116 may control the duty ratio of the voltage applied to the electromagnetic induction source 162. For example, the control unit 116 controls induction heating of the susceptor 161 by controlling the duty ratio of the voltage applied to the inverter circuit 164 in feedback control commensurate with the difference between the estimated temperature and the target temperature of the susceptor 161. This configuration allows the temperature of the susceptor 161 to be suitably controlled.

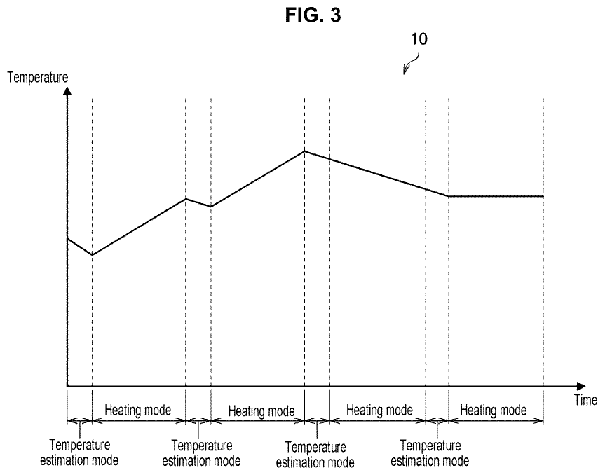

An example of the relationship between switching of the operating mode and estimating the temperature of the susceptor 161 will be described here with reference to FIG. 3. FIG. 3 is a diagram to illustrate an example of switching of the operating mode of the inhalation device 100 according to the embodiment. The vertical axis in a graph 10 shown in FIG. 3 denotes temperature of the susceptor 161, and the horizontal axis denotes time. As shown in FIG. 3, the control unit 116 repeatedly switches between the temperature estimation mode and the heating mode in accordance with elapsed time. Based on the temperature of the susceptor 161 estimated in the temperature estimation mode, the control unit 116 then raises, lowers or maintains the temperature of the susceptor 161 in the subsequent heating mode.

The power supplied to the resonant circuit 160 in the temperature estimation mode may be smaller than the power supplied to the resonant circuit 160 in the heating mode. As an example, the voltage applied to the resonant circuit 160 in the temperature estimation mode may be lower than the voltage applied to the resonant circuit 160 in the heating mode. This configuration makes it possible to lessen adverse effects on the temperature of the susceptor 161 caused by the temperature estimation processing, such as an unintended rise in the temperature of the susceptor 161 when a voltage is applied to the resonant circuit 160 in order to estimate the temperature of the susceptor 161. As a result, the temperature of the susceptor 161 can be controlled even more accurately.

However, the small amount of power supplied to the resonant circuit in the temperature estimation mode may cause a drop in the temperature of the susceptor 161 in the temperature estimation mode, as shown in the graph 10. For this reason, the duration of the temperature estimation mode is preferably shorter than the duration of the heating mode, as shown in FIG. 3. This configuration makes it possible to reduce the extent of a drop in the temperature of the susceptor 161 in the temperature estimation mode. By this means, the temperature of the susceptor 161 can be suitably raised at the timing at which the temperature of the susceptor 161 should be raised. In particular, the temperature of the susceptor 161 can be rapidly raised in the preheating period, which enables the preheating period to be shortened.

The control unit 116 may variably set the duration of the heating mode. As an example, the control unit 116 may set a longer duration of the heating mode for a greater difference between the estimated temperature and the target temperature of the susceptor 161. This makes it possible to promptly eliminate the difference between the estimated temperature and the target temperature of the susceptor 161. As another example, the control unit 116 may set a shorter duration of the heating mode for a smaller difference between the estimated temperature and the target temperature of the susceptor 161. This allows the temperature of the susceptor 161 to be estimated more frequently, therefore enabling the estimated temperature of the susceptor 161 to be brought closer to the target temperature by small degrees. This configuration thus allows the temperature of the susceptor 161 to be controlled even more accurately.

### (4) Control of Drive Frequency

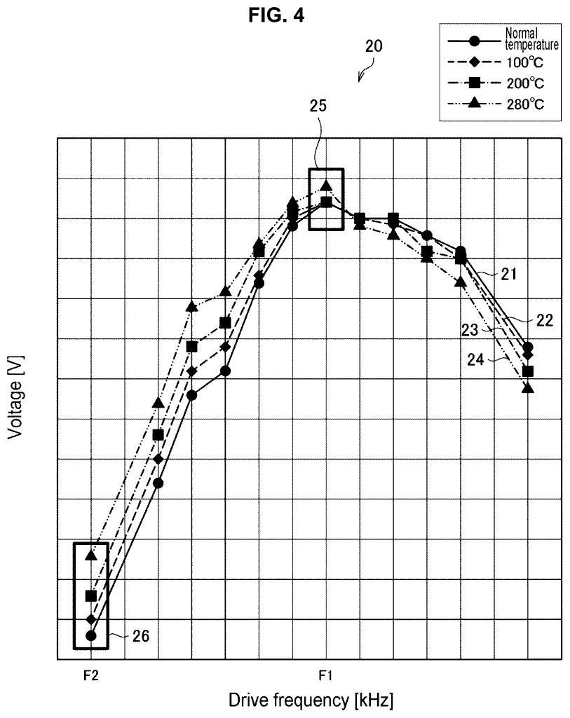

The present inventors carried out experiments to investigate the relationship between drive frequency of the resonant circuit 160 and accuracy of estimating the temperature of the susceptor 161. More specifically, the present inventors placed a susceptor 161 fitted with a heater at the same position as the susceptor 161 when the stick-type substrate 150 is accommodated in the accommodating portion 140. The present inventors then heated the susceptor 161 to a predetermined temperature by means of the heater, after which they measured the voltage of the capacitor 163 by means of an oscilloscope connected to both sides of the capacitor 163 while varying the drive frequency of the resonant circuit 160 by 2 kHz at a time. It should be noted that the present inventors used two types of resonant circuits 160 having different resonant frequencies. The capacitors 163 in these resonant circuits 160 had different capacitances. The results of the experiments will be described in detail below with reference to FIG. 4-7.

### Experimental Results in 300-400 kHz Band

FIG. 4 is a graph showing the relationship between the voltage of the capacitor 163 and the drive frequency in the resonant circuit 160 with a resonant frequency in the 300-400 kHz band. The vertical axis of a graph 20 denotes the voltage of the capacitor 163, which increases from bottom to top. The voltage as referred to here indicates the maximum value of the AC voltage of the capacitor 163. The horizontal axis of the graph 20 denotes the drive frequency of the resonant circuit 160, which increases from left to right. The line 21 shows the relationship between the voltage of the capacitor 163 and the drive frequency of the resonant circuit 160 when the temperature of the susceptor 161 is normal temperature. The line 22 shows the relationship between the voltage of the capacitor 163 and the drive frequency of the resonant circuit 160 when the temperature of the susceptor 161 is approximately 100° C. The line 23 shows the relationship between the voltage of the capacitor 163 and the drive frequency of the resonant circuit 160 when the temperature of the susceptor 161 is approximately 200° C. The line 24 shows the relationship between the voltage of the capacitor 163 and the drive frequency of the resonant circuit 160 when the temperature of the susceptor 161 is approximately 280° C.

It can be seen by referring to a region 25 in the graph 20 that when the drive frequency of the resonant circuit 160 is close to F1 kHz (resonant frequency), the voltage of the capacitor 163 is largely unchanged, even if the temperature of the susceptor 161 is different. Meanwhile, it can be seen by referring to a region 26 in the graph 20 that when the drive frequency of the resonant circuit 160 is F2 kHz, which is outside the resonant frequency of F1 kHz, there is a large difference in the voltage of the capacitor 163 depending on the difference in the temperature of the susceptor 161. That is, when the resonant circuit 160 is operated at a frequency outside the resonant frequency, it can be said that the voltage of the capacitor 163 can clearly reflect the temperature of the susceptor 161.

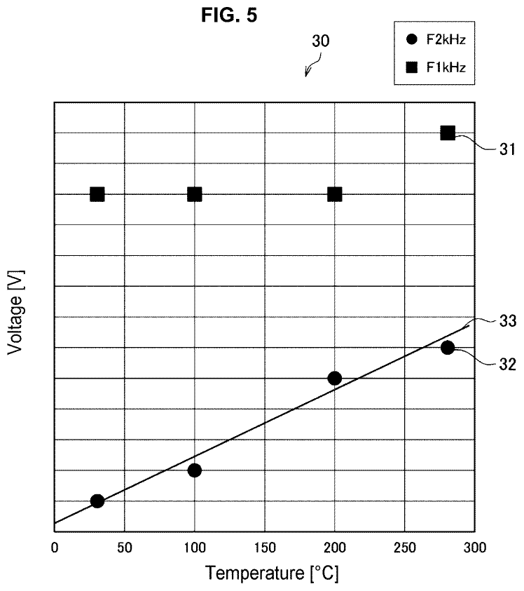

FIG. 5 is a graph showing the relationship between the voltage of the capacitor 163 and the temperature of the susceptor 161 when the resonant circuit 160 with a resonant frequency in the 300-400 kHz band is operated at the resonant frequency or a frequency outside the resonant frequency. The vertical axis of a graph 30 denotes the voltage of the capacitor 163, which increases from bottom to top. The voltage as referred to here indicates the maximum value of the AC voltage of the capacitor 163. The horizontal axis of the graph 30 denotes the temperature of the susceptor 161. The plots 31 show the relationship between the voltage of the capacitor 163 and the temperature of the susceptor 161 when the resonant circuit 160 is operated at F1 kHz (resonant frequency). The plots 32 show the relationship between the voltage of the capacitor 163 and the temperature of the susceptor 161 when the resonant circuit 160 is operated at F2 kHz, which is a frequency outside the resonant frequency. The line 33 is a regression line of the relationship between the voltage of the capacitor 163 and the temperature of the susceptor 161 when the resonant circuit 160 is operated at F2 kHz, which is a frequency outside the resonant frequency.

As shown by the plots 31 in the graph 30, when the resonant circuit 160 is operated at the resonant frequency, the voltage of the capacitor 163 is sometimes the same even if the temperature of the susceptor 161 is different. It would therefore be seen as difficult to estimate the temperature of the susceptor 161 based on the voltage of the capacitor 163. As shown by the plots 32 and the line 33 in the graph 30, on the other hand, when the resonant circuit 160 is operated at a frequency outside the resonant frequency, the voltage of the capacitor 163 and the temperature of the susceptor 161 can be said to have a linear relationship. To be more specific, the voltage of the capacitor 163 increases as the temperature of the susceptor 161 increases. In light of the above, the temperature of the susceptor 161 can be accurately estimated based on a lookup table defining this linear relationship when the resonant circuit 160 is operated at a frequency outside the resonant frequency.

### Experimental Results in 1000-1100 kHz Band

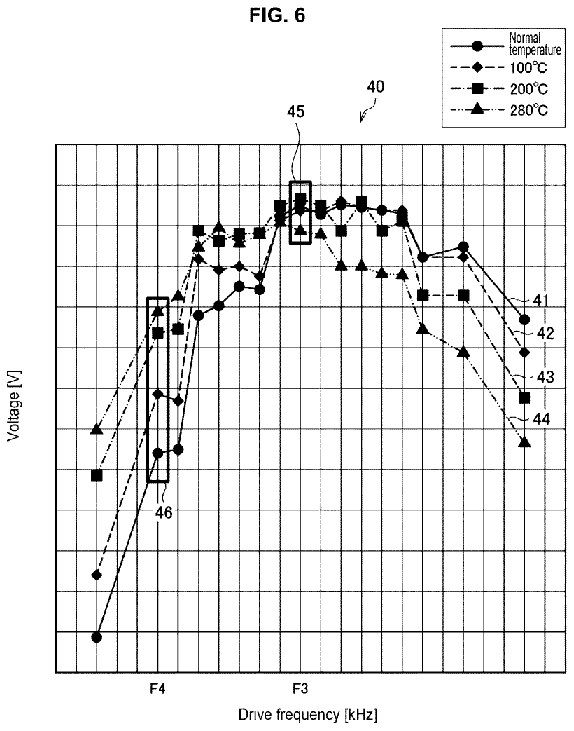

FIG. 6 is a graph showing the relationship between the voltage of the capacitor 163 and the drive frequency in the resonant circuit 160 with a resonant frequency in the 1000-1100 kHz band. The vertical axis of a graph 40 denotes the voltage of the capacitor 163, which increases from bottom to top. The voltage as referred to here indicates the maximum value of the AC voltage of the capacitor 163. The horizontal axis of the graph 40 denotes the drive frequency of the resonant circuit 160, which increases from left to right. The line 41 shows the relationship between the voltage of the capacitor 163 and the drive frequency of the resonant circuit 160 when the temperature of the susceptor 161 is normal temperature. The line 42 shows the relationship between the voltage of the capacitor 163 and the drive frequency of the resonant circuit 160 when the temperature of the susceptor 161 is approximately 100° C. The line 43 shows the relationship between the voltage of the capacitor 163 and the drive frequency of the resonant circuit 160 when the temperature of the susceptor 161 is approximately 200° C. The line 44 shows the relationship between the voltage of the capacitor 163 and the drive frequency of the resonant circuit 160 when the temperature of the susceptor 161 is approximately 280° C.

It can be seen by referring to a region 45 in the graph 40 that when the drive frequency of the resonant circuit 160 is close to F3 kHz (resonant frequency), the voltage of the capacitor 163 is largely unchanged, even if the temperature of the susceptor 161 is different. Meanwhile, it can be seen by referring to a region 46 in the graph 40 that when the drive frequency of the resonant circuit 160 is F4 kHz, which is outside the resonant frequency of F3 kHz, there is a large difference in the voltage of the capacitor 163 depending on the difference in the temperature of the susceptor 161. That is, when the resonant circuit 160 is operated at a frequency outside the resonant frequency, it can be said that the voltage of the capacitor 163 can clearly reflect the temperature of the susceptor 161.

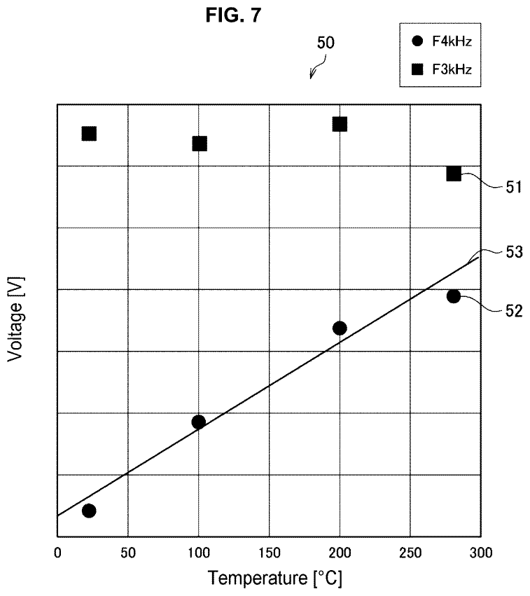

FIG. 7 is a graph showing the relationship between the voltage of the capacitor 163 and the temperature of the susceptor 161 when the resonant circuit 160 with a resonant frequency in the 1000-1100 kHz band is operated at the resonant frequency or a frequency outside the resonant frequency. The vertical axis of a graph 50 denotes the voltage of the capacitor 163, which increases from bottom to top. The voltage as referred to here indicates the maximum value of the AC voltage of the capacitor 163. The horizontal axis of the graph 50 denotes the temperature of the susceptor 161. The plots 51 show the relationship between the voltage of the capacitor 163 and the temperature of the susceptor 161 when the resonant circuit 160 is operated at F3 kHz (resonant frequency). The plots 52 show the relationship between the voltage of the capacitor 163 and the temperature of the susceptor 161 when the resonant circuit 160 is operated at F4 kHz, which is a frequency outside the resonant frequency. The line 53 is a regression line of the relationship between the voltage of the capacitor 163 and the temperature of the susceptor 161 when the resonant circuit 160 is operated at F4 kHz, which is a frequency outside the resonant frequency.

As shown by the plots 51 in the graph 50, a stable relationship is not observed between the temperature of the susceptor 161 and the voltage of the capacitor 163 when the resonant circuit 160 is operated at the resonant frequency. It would therefore be seen as difficult to estimate the temperature of the susceptor 161 based on the voltage of the capacitor 163. As shown by the plots 52 and the line 53 in the graph 50, on the other hand, when the resonant circuit 160 is operated at a frequency outside the resonant frequency, it can be said that the voltage of the capacitor 163 and the temperature of the susceptor 161 have a linear relationship. To be more specific, the voltage of the capacitor 163 increases as the temperature of the susceptor 161 increases. In light of the above, the temperature of the susceptor 161 can be accurately estimated based on a lookup table defining this linear relationship when the resonant circuit 160 is operated at a frequency outside the resonant frequency.

### Configuration of Inhalation Device 100 Based on Experimental Results

As described above, the control unit 116 repeatedly switches the operating mode of the resonant circuit 160 to the temperature estimation mode or the heating mode in the heating processing based on the heating profile. Here, the control unit 116 causes the resonant circuit 160 to operate at a frequency included in a first frequency band in the temperature estimation mode. Meanwhile, the control unit 116 causes the resonant circuit 160 to operate at a frequency included in a second frequency band in the heating mode. The first frequency band and the second frequency band are separate from each other. In other words, there is no overlap between the first frequency band and the second frequency band. This configuration allows the resonant circuit 160 to be driven at a suitable drive frequency for each of the temperature estimation mode and the heating mode. As a result, the quality of experience for a user employing the induction-heating inhalation device 100 can be further improved.

The first frequency band does not include the resonant frequency of the resonant circuit 160. That is to say, in the temperature estimation mode, the control unit 116 drives the resonant circuit 160 at a frequency outside the resonant frequency. This configuration makes it possible to improve the accuracy of estimating the temperature of the susceptor 161 in the temperature estimation mode.

The first frequency band may be a lower frequency band than the second frequency band. As an example, when the inhalation device 100 comprises the resonant circuit 160 used in the experiments shown in FIGS. 4 and 5, the control unit 116 may use, as the first frequency band, a frequency band around F2 kHz, which is lower than the resonant frequency of F1 kHz. As another example, when the inhalation device 100 comprises the resonant circuit 160 used in the experiments shown in FIGS. 6 and 7, the control unit 116 may use, as the first frequency band, a frequency band around F4 kHz, which is lower than the resonant frequency of F3 kHz. This configuration makes it possible to improve the accuracy of estimating the temperature of the susceptor 161 in the temperature estimation mode, as described with reference to FIG. 4-7.

Meanwhile, the second frequency band includes the resonant frequency of the resonant circuit 160. That is to say, in the heating mode, the control unit 116 drives the resonant circuit 160 at a frequency in the region of the resonant frequency. This configuration makes it possible to suppress power losses in the electromagnetic induction source 162 and the capacitor 163. As a result, the susceptor 161 can be efficiently inductively heated in the heating mode.

As an example, when the inhalation device 100 comprises the resonant circuit 160 used in the experiments shown in FIGS. 4 and 5, the control unit 116 may use, as the second frequency band, a frequency band around the resonant frequency of F1 kHz. As another example, when the inhalation device 100 comprises the resonant circuit 160 used in the experiments shown in FIGS. 6 and 7, the control unit 116 may use, as the second frequency band, a frequency band around the resonant frequency of F3 kHz.

The resonant frequency of the resonant circuit 160 may fluctuate depending on the temperature of the susceptor 161. From this perspective, the second frequency band preferably includes the resonant frequency of the resonant circuit 160 when the temperature of the susceptor 161 is a temperature which would be expected in the heating processing based on the heating profile. More specifically, the second frequency band preferably includes the resonant frequency of the resonant circuit 160 when the temperature of the susceptor 161 is the highest temperature expected in the heating processing based on the heating profile. For example, if the highest target temperature defined in the heating profile is 300° C., then the second frequency band preferably includes the resonant frequency of the resonant circuit 160 when the temperature of the susceptor 161 is 300° C. Furthermore, the second frequency band preferably includes the resonant frequency of the resonant circuit 160 when the temperature of the susceptor 161 is the lowest temperature expected in the heating processing based on the heating profile. Put more simply, the second frequency band preferably includes the resonant frequency of the resonant circuit 160 when the temperature of the susceptor 161 is the temperature before the heating processing based on the heating profile is implemented, i.e., normal temperature. Furthermore, in terms of the first frequency band, the first frequency band preferably does not include the resonant frequency of the resonant circuit 160 when the temperature of the susceptor 161 is a temperature which would be expected in the heating processing based on the heating profile. This configuration makes it possible to achieve a balance between improving the accuracy of estimating the temperature of the susceptor 161 in the temperature estimation mode, and improving the efficiency of heating the susceptor 161 in the heating mode, at any timing while heating based on the heating profile is in progress.

In the temperature estimation mode, the control unit 116 may use one fixed frequency included in the first frequency band. The control unit 116 may especially use a fixed frequency outside the resonant frequency region in the temperature estimation mode. It should be noted that the resonant frequency region may be a frequency included in a range of ±15 kHz from the resonant frequency. The resonant frequency region may preferably be a frequency included in a range of ±10 kHz from the resonant frequency, may more preferably be a frequency included in a range of ±5 kHz from the resonant frequency, and may even more preferably be a frequency included in a range of ±2 kHz from the resonant frequency. Furthermore, a frequency outside the resonant frequency may be a frequency deviating by at least 5 kHz, preferably at least 10 kHz, and more preferably at least 15 kHz from the resonant frequency. As an example, when the inhalation device 100 comprises the resonant circuit 160 used in the experiments shown in FIGS. 4 and 5, the control unit 116 may use F2 kHz, which is a frequency outside the resonant frequency, in the temperature estimation mode. As another example, when the inhalation device 100 comprises the resonant circuit 160 used in the experiments shown in FIGS. 6 and 7, the control unit 116 may use F4 kHz, which is a frequency outside the resonant frequency, in the temperature estimation mode. This configuration makes it possible to improve the accuracy of estimating the temperature of the susceptor 161.

In the heating mode, the control unit 116 may use one fixed frequency included in the second frequency band. The control unit 116 may especially use a frequency in the resonant frequency region or the resonant frequency itself in a fixed manner in the heating mode. As an example, when the inhalation device 100 comprises the resonant circuit 160 used in the experiments shown in FIGS. 4 and 5, the control unit 116 may use the resonant frequency of F1 kHz in the heating mode. As another example, when the inhalation device 100 comprises the resonant circuit 160 used in the experiments shown in FIGS. 6 and 7, the control unit 116 may use the resonant frequency of F3 kHz in the heating mode. Power losses in the electromagnetic induction source 162 and the capacitor 163 can be minimized by driving the resonant circuit 160 in the resonant frequency region. As a result, the susceptor 161 can be inductively heated most efficiently in the heating mode.

It should be noted that the resonant frequency of the resonant circuit 160 may be acquired as a factory default for the inhalation device 100. The drive frequencies used in each of the temperature estimation mode and the heating mode can then be set based on the factory default resonant frequency.

### (5) Processing Flow

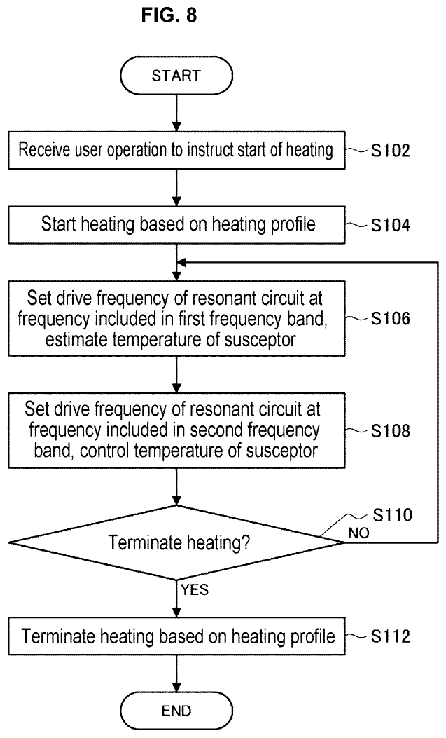

FIG. 8 is a flowchart showing an example of a flow of processing implemented by the inhalation device 100 according to the embodiment.

As shown in FIG. 8, the inhalation device 100 first of all receives a user operation for instructing the start of heating (step S102). As an example, the control unit 116 may receive a user operation of pressing a predetermined button as the user operation for instructing the start of heating. As another example, the control unit 116 may receive a user operation of inserting the stick-type substrate 150 into the accommodating portion 140 as the user operation for instructing the start of heating.

The inhalation device 100 then starts heating based on the heating profile (step S104). For example, the control unit 116 starts applying a voltage from the power source unit 111 to the resonant circuit 160.

The inhalation device 100 then operates in the temperature estimation mode. That is to say, the inhalation device 100 sets the drive frequency of the resonant circuit 160 at a frequency included in the first frequency band, and estimates the temperature of the susceptor 161 (step S106). For example, the control unit 116 drives the resonant circuit 160 at a frequency outside the resonant frequency and estimates the temperature of the susceptor 161 based on the voltage of the capacitor 163.

The inhalation device 100 then operates in the heating mode. That is to say, the inhalation device 100 sets the drive frequency of the resonant circuit 160 at a frequency included in the second frequency band, and controls the temperature of the susceptor 161 (step S108). For example, the control unit 116 controls the duty ratio of the voltage applied to the resonant circuit 160 in such a way as to narrow the difference between the estimated temperature and the target temperature of the susceptor 161, while driving the resonant circuit 160 at a frequency in the resonant frequency region.

The inhalation device 100 then determines whether or not to terminate heating (step S110). As an example, the control unit 116 determines that heating is to be terminated when the elapsed time since the start of heating based on the heating profile has reached a predetermined threshold. As another example, the control unit 116 determines that heating is to be terminated when the number of puffs has reached a predetermined threshold.

If it is determined that heating is not to be terminated (step S110: NO), the processing once again returns to step S106.

Meanwhile, if it is determined that heating is to be terminated (step S110: YES), the inhalation device 100 terminates heating based on the heating profile (step S112). For example, the control unit 116 stops applying a voltage from the power source unit 111 to the resonant circuit 160.

### 3. Supplementary Information

A preferred embodiment of the present disclosure has been described in detail above with reference to the appended drawings, but the present disclosure is not limited to such an example. It is obvious that a person having ordinary knowledge in the technical field to which the present disclosure belongs will be able to conceive of a number of variant examples or modified examples within the scope of the technical concept disclosed in the claims, and any such variant examples or modified examples are naturally understood to fall within the technical scope of the present disclosure.

The embodiment above described an example in which the first frequency band is a lower frequency band than the second frequency band, but the present disclosure is not limited to such an example. The first frequency band may equally be a higher frequency band than the second frequency band, provided that it is separate from the second frequency band. It can be seen by referring to FIG. 4 that there is a large difference in the voltage of the capacitor 163 depending on the difference in the temperature of the susceptor 161 not only in lower frequency bands than the resonant frequency of F1 kHz, but also in higher frequency bands. Similarly, it can be seen by referring to FIG. 6 that there is a large difference in the voltage of the capacitor 163 depending on the difference in the temperature of the susceptor 161 not only in lower frequency bands than the resonant frequency of F3 kHz, but also in higher frequency bands. This means that it is also possible to accurately estimate the temperature of the susceptor 161 based on the voltage of the capacitor 163 when a frequency band higher than the second frequency band is used as the first frequency band.

The control unit 116 may switch the first frequency band to a higher frequency band or a lower frequency band than the second frequency band, based on electrical characteristics of the resonant circuit 160 acquired in the temperature estimation mode. Put more simply, the control unit 116 may switch the first frequency band to a higher frequency band or a lower frequency band than the second frequency band, based on the temperature of the susceptor 161 estimated in the temperature estimation mode. As an example, the resonant frequency of the resonant circuit 160 might be expected to fall as the temperature of the susceptor 161 increases. In that case, the control unit 116 may use a lower frequency band than the second frequency band as the first frequency band when the estimated temperature of the susceptor 161 is lower than a predetermined threshold, and may use a higher frequency band than the second frequency band as the first frequency band when the estimated temperature of the susceptor 161 is higher than the predetermined threshold. If the resonant frequency of the resonant circuit 160 is expected to rise as the temperature of the susceptor 161 increases, then the control unit 116 should implement control in the opposite way to that described above. By virtue of this configuration, the drive frequency of the resonant circuit 160 in the temperature estimation mode can be maintained at a frequency far from the resonant frequency of the resonant circuit 160, even if the resonant frequency of the resonant circuit 160 changes in response to a change in the temperature of the susceptor 161. As a result, it is possible to improve the accuracy of estimating the temperature of the susceptor 161 in the temperature estimation mode.

The embodiment above described an example in which the resonant circuit 160 is configured as a series resonant circuit in which the electromagnetic induction source 162 and the capacitor 163 are connected in series, but the present disclosure is not limited to such an example. The resonant circuit 160 may equally be configured as a parallel resonant circuit in which the electromagnetic induction source 162 and the capacitor 163 are connected in parallel.

The embodiment above described an example in which the inverter circuit 164 is a half-bridge inverter, but the present disclosure is not limited to such an example. As an example, the inverter circuit 164 may be a full-bridge inverter. As another example, the inverter circuit 164 may be a single-ended circuit such as a class-E amplifier. More specifically, the inverter circuit 164 may be a single-ended circuit which includes an N-channel FET but does not include a P-channel FET, for example.

The inhalation device 100 described above is an example of an aerosol-generating system which generates an aerosol to be inhaled by a user by using a substrate containing either one of an aerosol source and a flavor source. The flavor source is a component for imparting a flavor component to the aerosol. The stick-type substrate 150 is an example of a substrate used in the aerosol-generating system. The combination of the inhalation device 100 and the stick-type substrate 150 may also be seen as an aerosol-generating system.

The stick-type substrate 150 was cited as an example of an aerosol source-containing substrate in the embodiment above, but the substrate may have any shape. For example, the aerosol source-containing substrate may be formed with a cup shape or a flat shape. A cup-shaped substrate may be formed by filling a hollow container of any shape with the aerosol source, for example. A flat substrate may be formed by forming the aerosol source into a thin, planar shape, for example.

It should be noted that the series of processes performed by each device described in the present description may be realized by using software, hardware, or any combination of software and hardware. Programs constituting the software are prestored on a recording medium (more specifically, a non-transitory computer-readable storage medium) provided internally or externally to each device, for example. When the programs are then executed, for example, by a computer for controlling each device described in the present description, the programs are read into a RAM and executed by means of a processing circuit such as a CPU. The recording medium is, for example, a magnetic disk, an optical disk, a magneto-optical disk, or a flash memory, etc. Furthermore, the computer programs may be distributed via a network, for example, without the use of a recording medium. Furthermore, the computer may be an application-specific integrated circuit such as ASIC, a general-purpose processor which executes functions by reading software programs, or a computer on a server used for cloud computing, etc. Furthermore, the series of processes performed by each device described in the present description may be processed centrally by a single computer, or may be processed in a distributed manner by multiple computers. In addition, in the embodiments described above, two or more communication means present in a single device may be physically realized by a single medium.

Furthermore, the processing described using flowcharts or sequence diagrams in the present description need not necessarily be implemented in the order depicted. Some processing steps may be implemented in parallel. Furthermore, additional processing steps may be employed and some processing steps may be omitted.

The following configurations also fall within the technical scope of the present disclosure.
(1) An aerosol-generating system comprising: an accommodating portion for accommodating a substrate containing an aerosol source;
a resonant circuit comprising an electromagnetic induction source for inductively heating a susceptor arranged thermally adjacent to the aerosol source of the substrate accommodated in the accommodating portion; and
a control unit for controlling operation of the resonant circuit so as to implement processing to generate an aerosol using the substrate,
wherein
the control unit repeatedly switches an operating mode of the resonant circuit to a first operating mode or a second operating mode in the processing to generate an aerosol using the substrate,
the first operating mode comprises causing the resonant circuit to operate at a frequency included in a first frequency band,
the second operating mode comprises causing the resonant circuit to operate at a frequency included in a second frequency band, and
the first frequency band and the second frequency band are separate from each other.
(2) The aerosol-generating system as disclosed in (1) above, wherein the second frequency band includes a resonant frequency of the resonant circuit when a temperature of the susceptor is the highest temperature expected in the processing to generate an aerosol using the substrate.
(3) The aerosol-generating system as disclosed in (1) or (2) above, wherein the second frequency band includes a resonant frequency of the resonant circuit when a temperature of the susceptor is the lowest temperature expected in the processing to generate an aerosol using the substrate.
(4) The aerosol-generating system as disclosed in any one of (1) to (3) above, wherein the first frequency band is a higher frequency band or a lower frequency band than the second frequency band.
(5) The aerosol-generating system as disclosed in any one of (1) to (4) above, wherein, in the first operating mode, the control unit uses one fixed frequency included in the first frequency band.
(6) The aerosol-generating system as disclosed in any one of (1) to (5) above, wherein, in the second operating mode, the control unit uses one fixed frequency included in the second frequency band.
(7) The aerosol-generating system as disclosed in (4) above, wherein the control unit switches the first frequency band to a higher frequency band or a lower frequency band than the second frequency band, based on electrical characteristics of the resonant circuit acquired in the first operating mode.
(8) The aerosol-generating system as disclosed in any one of (1) to (7) above, wherein the control unit controls operation of the resonant circuit in the second operating mode based on electrical characteristics of the resonant circuit acquired in the first operating mode.
(9) The aerosol-generating system as disclosed in (8) above, wherein the resonant circuit further comprises a capacitor, and
the control unit controls operation of the resonant circuit in the second operating mode based on a voltage of the capacitor acquired in the first operating mode.
(10) The aerosol-generating system as disclosed in (9) above, wherein the control unit estimates a temperature of the susceptor based on the voltage of the capacitor acquired in the first operating mode, and controls operation of the resonant circuit in the second operating mode based on the estimated temperature of the susceptor and a predefined target temperature of the susceptor.
(11) The aerosol-generating system as disclosed in any one of (8) to (10) above, wherein, in the second operating mode, the control unit controls a duty ratio of a voltage applied to the resonant circuit.
(12) The aerosol-generating system as disclosed in any one of (1) to (11) above, wherein power supplied to the resonant circuit in the first operating mode is smaller than power supplied to the resonant circuit in the second operating mode.
(13) The aerosol-generating system as disclosed in any one of (1) to (12) above, wherein a duration of the first operating mode is shorter than a duration of the second operating mode.
(14) The aerosol-generating system as disclosed in any one of (1) to (13) above, wherein the control unit variably sets the duration of the second operating mode.
(15) The aerosol-generating system as disclosed in any one of (1) to (14) above, further comprising the substrate, wherein the substrate further contains the susceptor.

### REFERENCE SIGNS LIST

100 Inhalation device
111 Power source unit
112 Sensor unit
113 Notification unit
114 Memory unit
115 Communication unit
116 Control unit
140 Accommodating portion
141 Internal space
142 Opening
143 Bottom portion
150 Stick-type substrate
151 Substrate portion
152 Mouthpiece portion
160 Resonant circuit
161 Susceptor
162 Electromagnetic induction source
163 Capacitor
164 Inverter circuit
165 FET

1.-15. (canceled)

16. An aerosol-generating system comprising:
a chamber configured to accommodate a substrate containing an aerosol source;
a resonant circuit comprising an electromagnetic induction source configured to inductively heat a susceptor arranged thermally adjacent to the aerosol source of the substrate accommodated in the chamber; and
circuitry configured to repeatedly switch an operating mode of the resonant circuit between a first operating mode and a second operating mode to generate aerosol using the substrate, wherein
the first operating mode comprises the circuitry causing the resonant circuit to operate at a first frequency included in a first frequency band, and
the second operating mode comprises the circuitry causing the resonant circuit to operate at a second frequency included in a second frequency band, which is different from the first frequency band.

17. The aerosol-generating system of claim 16, wherein
the second frequency band includes a resonant frequency of the resonant circuit when a temperature of the susceptor is above a predetermined threshold.

18. The aerosol-generating system of claim 16, wherein
the second frequency band includes a resonant frequency of the resonant circuit when a temperature of the susceptor is below a predetermined threshold.

19. The aerosol-generating system of claim 16, wherein
the first frequency band is a higher frequency band or a lower frequency band than the second frequency band.

20. The aerosol-generating system of claim 16, wherein
in the first operating mode, the circuitry is configured to use one fixed frequency included in the first frequency band.

21. The aerosol-generating system of claim 16, wherein
in the second operating mode, the circuitry is configured to use one fixed frequency included in the second frequency band.

22. The aerosol-generating system of claim 19, wherein
the circuitry is configured to switch the first frequency band to a higher frequency band or a lower frequency band than the second frequency band, based on electrical characteristics of the resonant circuit acquired in the first operating mode.

23. The aerosol-generating system of claim 16, wherein
the circuitry is configured to control operation of the resonant circuit in the second operating mode based on electrical characteristics of the resonant circuit acquired in the first operating mode.

24. The aerosol-generating system of claim 23, wherein
the resonant circuit comprises a capacitor, and
the circuitry is configured to control operation of the resonant circuit in the second operating mode based on a voltage of the capacitor acquired in the first operating mode.

25. The aerosol-generating system of claim 24, wherein the circuitry is configured to:
estimate a temperature of the susceptor based on the voltage of the capacitor acquired in the first operating mode; and
control operation of the resonant circuit in the second operating mode based on the estimated temperature of the susceptor and a predefined target temperature of the susceptor.

26. The aerosol-generating system of claim 23, wherein
in the second operating mode, the circuitry is configured to control a duty ratio of a voltage applied to the resonant circuit.

27. The aerosol-generating system of claim 16, wherein
power supplied to the resonant circuit in the first operating mode is smaller than power supplied to the resonant circuit in the second operating mode.

28. The aerosol-generating system of claim 16, wherein
a duration of the first operating mode is shorter than a duration of the second operating mode.

29. The aerosol-generating system of claim 16, wherein
the circuitry is configured to variably set the duration of the second operating mode.

30. The aerosol-generating system of claim 16, further comprising:
the substrate, wherein the substrate further contains the susceptor.

31. The aerosol-generating system of claim 16, further comprising:
means for outputting a notification to a ser of the aerosol-generating system.

32. The aerosol-generating system of claim 16, further comprising:
a sensor configured to acquire values associated with inhalation by a user.

33. The aerosol-generating system of claim 16, wherein
the electromagnetic induction source comprises a coiled conductor disposed around an outer periphery of the chamber.

34. An aerosol-generating system comprising:
a chamber configured to accommodate a substrate containing an aerosol source;
an electromagnetic induction source configured to inductively heat a susceptor arranged thermally adjacent to the aerosol source of the substrate accommodated in the chamber; and
circuitry configured to control operation of the electromagnetic induction source to generate aerosol using the substrate by operating the electromagnetic induction source at different frequencies during aerosol generation.

35. An aerosol-generating system comprising:
a chamber configured to accommodate a substrate containing an aerosol source;
a resonant circuit comprising an electromagnetic induction source configured to inductively heat a susceptor arranged thermally adjacent to the aerosol source of the substrate accommodated in the chamber and a capacitor;
circuitry configured to
repeatedly switch an operating mode of the resonant circuit between a first operating mode and a second operating mode to generate aerosol using the substrate, wherein the first operating mode comprises the circuitry causing the resonant circuit to operate at a first frequency included in a first frequency band that does not include a resonant frequency of the resonant circuit, and the second operating mode comprises the circuitry causing the resonant circuit to operate at a second frequency included in a second frequency band that includes the resonant frequency of the resonant circuit;
estimate a temperature of the susceptor based on a voltage of the capacitor acquired in the first operating mode; and
control a duty ratio of a voltage applied to the resonant circuit in the second operating mode based on the estimated temperature.
